(19) United States
(12) Patent Application Publication
(10) Pub. No.: US 2011/0009536 A1
(43) Pub. Date: Jan. 13, 2011

(54) C.I. PIGMENT VIOLET 37 IN ROD-LIKE FORM

(75) Inventors: Marc Maurer, Village-Neuf (FR); Niklaus Raetzo, Reinach (CH)

Correspondence Address: BASF Corporation;Patent Department, 500 White Plains Road, P.O. Box 2005, Tarrytown, NY, 10591, US

(73) Assignee: CIBA CORPORATION, Tarrytown, NY (US)

(21) Appl. No.: 12/517,888

(22) PCT Filed: Dec. 4, 2007
(86) PCT No.: PCT/EP2007/063208

§ 371 Date: Sep. 17, 2010

(30) Foreign Application Priority Data
Dec. 14, 2006 (EP) 06126131.9

(51) Int. Cl. C08K 5/3437; C07D 265/34; B32B 1/00; C09D 11/10

(52) U.S. Cl. 524/90; 544/99; 428/220

(57) ABSTRACT

The present invention is directed to a new C.I. Pigment Violet 37 in rod-like form, a process for its preparation and its use for coloring high molecular weight organic material. The C.I. Pigment Violet 37 in rod-like form having a length of 0.5 to 30 μm, a width of 0.05 to 1 μm, especially 0.100 to 0.500 μm and an average thickness of 0.01 to 0.200 μm, especially 0.03 to 0.100 μm.

The present invention is directed to a new C.I. Pigment Violet 37 in rod-like form

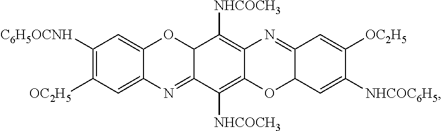

a process for its preparation and its use for coloring high molecular weight organic material.

U.S. Pat. No. 5,298,076 discloses a process for coloring a high molecular weight organic material which comprises incorporating an effective pigmenting amount of carbazole dioxazine crude pigment, i.e. C.I. Pigment Violet 23, having a specific surface area of from about 2 to 20 m²/g and an average particle size of 0.5 to 20 μm, into a high molecular weight organic material. The process is especially useful for producing multicolor effects and camouflage effects and for use in automotive coatings.

DE102005008659 relates to a process for the preparation of a transparent dioxazine pigment, such as C.I. Pigment Violet 37, which comprises milling a raw dioxazine pigment in eccentric oscillating mill with at least a grinding container stored at oscillating element and with an exciter unit fastened rigidly to the grinding container.

EP1199309A1 relates to a process for phase conversion of a methyl-substituted, benzimidazolone-fused dioxazine pigment of composition C₂₂H₁₂Cl₂N₆O₄ having the formula

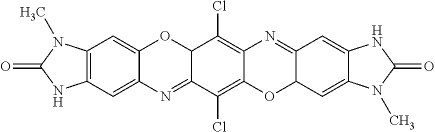

or of an isomer or tautomer thereof, which comprises treating the pigment of formula (1) with certain organic solvents. In the course of this phase conversion, 4 novel crystal polymorphs are formed which are called phases II, IV, V and VI and are characterized by means of their X-ray powder diagrams.

WO9845757 relates to a black-pigmented high molecular weight organic material structured from a radiation-sensitive precursor by irradiation, the pigmentation of which material consists of coloured organic pigments, at least one of which is in latent form before irradiation.

In Example 5 of WO9845757 the synthesis of the following compound

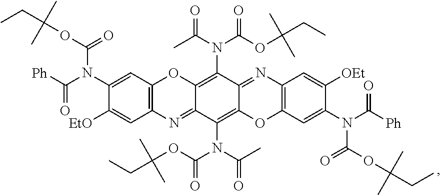

i.e. a radiation-sensitive precursor of C.I. Pigment Violet 37, is described, which is used for coloring high molecular weight organic materials. That is, a thin film comprising the precursor is obtained by spin-coating a glass plate. The film is dried and the precursor is converted to C.I. Pigment Violet 37 by UV light.

U.S. Pat. No. 3,472,844 relates to a new red-violet crystal form (the “(β-modification”) of 2,6-benzoylamino-3,7-diethoxy-9,10-diacetylamino-triphendioxazine of the formula

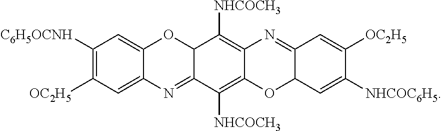

It may be obtained by heating 2,5-diacetylamino-3,6-di-(2′,5′-diethoxy-4′-benzoylamino)-1,4-benzoquinone for a prolonged period above 150° C. in nitrobenzene in the presence of an additive consisting of a base and/or water, or by heating the “α-modification” of 2,6-benzoylamino-3,7-diethoxy-9,10-diacetylamino-triphendioxazine for a prolonged period above 160° C. in nitrobenzene.

JP56135556 describes a process for the preparation of dioxazine compounds of formula

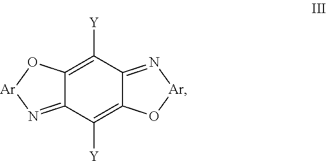

e.g. a compound of formula

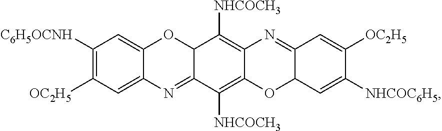

comprising heating a diimide compound
of formula I

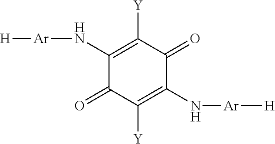

[wherein Ar is a (substituted) aromatic residue; Y is halogen, acylamino or acyloxy], at 100-200° C. in a polar, aprotic solvent. The pigment obtained by finely dividing said dioxazine compound is more excellent in bleed resistance as compared with the conventional one.

According to Practical Example 3 green crystals of C.I. Pigment Violet 37 are obtained by heating

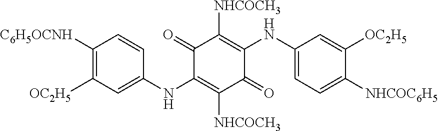

in the presence of p-toluenesulphonyl chloride at 150 to 160° C. in quinoline.

It has now been surprisingly discovered that crude C.I. Pigment Violet 37, known to exist in an isometric particle form having a particle size of 0.5 to 1 μm, can be readily and efficiently converted into an “effect” pigment having a rod-like form by recrystallising the crude C.I. Pigment Violet 37 at high temperature in polar solvents.

Accordingly, the present invention relates to a C.I. Pigment Violet 37 in rod-like form. The C.I. Pigment Violet 37 in rod-like form has a length of 0.5 to 30 μm, especially 1 to 30 μm, a width of 0.05 to 1 μm, especially 0.100 to 0.500 μm and an average thickness of 0.01 to 0.200 μm, especially 0.03 to 0.100 μm. New colouristic-properties are obtained with the C.I. Pigment Violet 37, such as, for example, different colours in remission and transmission or developing different colours depending on the viewing angle. Hence, it is predestinated for use as effect pigment, for example, in security applications.

In contrast to the traditional application fields for this pigment (particle size ˜50 nm), the new pigment is only interesting in its new and very big crystal dimension.

In addition, the present invention is directed to a process for the preparation of the C.I. Pigment Violet 37 in rod-like form, which comprises

(a) dispersing and partially dissolving crude C.I. Pigment Violet 37 in a polar solvent at elevated temperatures for a defined period of time.

Usually, the temperature of the mixture is lowered to 20 to 40° C. to inhibit further crystallisation.

If incorporated in 1 mm rolled PVC sheets as illustrated in application example 1, the C.I. Pigment Violet 37 in rod-like form obtainable according to the invention (1.0% pigment) is characterized in that
the colour in transmission is violet,
the colour in remisson is varying form green to brown and then black depending on the crystal length.

That means, by varying the residence time of the pigment in NMP (form 1 minute to 12 hours) the new colouristic-properties are obtained, i.e. the colour in remisson is varying form green to brown and then black depending on the crystal length. In all cases the colour in transmission is violet.

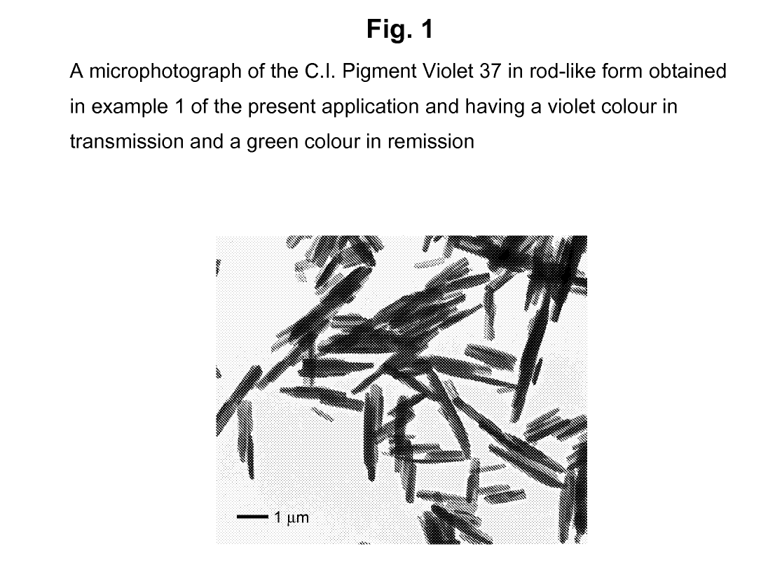

FIG. 1 is a microphotograph of the C.I. Pigment Violet 37 in rod-like form obtained in example 1 of the present application and having a violet colour in transmission and a green colour in remission.

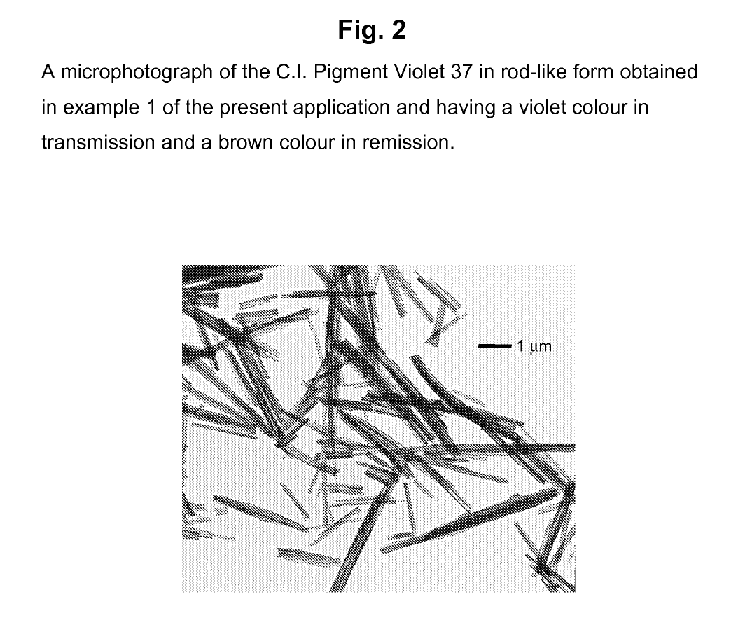

FIG. 2 is a microphotograph of the C.I. Pigment Violet 37 in rod-like form obtained in example 1 of the present application and having a violet colour in transmission and a brown colour in remission.

The novel process for preparation of the rod-like form of C.I. Pigment Violet 37 is conducted by dispersing and partially dissolving crude C.I. Pigment Violet 37 in a polar solvent at elevated temperatures. Elevated temperatures mean temperatures from 100° C. to reflux temperature of the solvent, especially from 120° C. to reflux temperature of the solvent. The C.I. Pigment Violet 37 in the desired rod-like form is fully obtained by maintaining the temperature at a certain level for a defined time (preferably 120° C. to reflux and 1 minute to 12 hours). Afterwards the temperature is lowered, preferably to 0 to 50° C., especially to 20 to 40° C., whereby further crystallisation is inhibited. The suspension is then cooled to room temperature, filtered, the filtrate is washed with water and the pigment is dried.

The starting C.I. Pigment Violet 37 crude is a conventional commercially available isometric C.I. Pigment Violet 37 crude having a particle size of 0.5 to 1 μm.

Suitable polar solvents are N,N,N′,N′-tetramethylurea, N,N-dimethylacetamide, N-methylformamide and N,N-dimethylformamide (DMF) and N-methyl-pyrrolidone (NMP). N,N-dimethylformamide and N-methyl-pyrrolidone are preferred and N-methyl-pyrrolidone is most preferred.

The molar ratio of the C.I. Pigment Violet 37 crude to solvent is 1:270 to 1:27, especially 1:133 to 1:33.

The amount of solvent is dependent on the polarity of the solvent, i.e. the solubility of C.I. Pigment Violet 37 in the solvent.

If the solvent is NMP, NMP is present in an amount of 3 to 30 parts by weight, especially by 4 to 20 parts by weight, per part of pigment.

The novel process for preparation of the rod-like form of C.I. Pigment Violet 37 is illustrated in more detail with respect to NMP as solvent, but is not limited thereto.

A mixture of NMP and C.I. Pigment Violet 37 crude is heated to a temperature of from about 170° C. to reflux temperature of the NMP and stirred for 1 minute to 12 hours at said temperature. The temperature of the mixture is lowered to 20 to 40° C. to inhibit further crystallisation. The suspension is then cooled to room temperature, filtered, the filtrate is washed with water to remove the NMP and the pigment is dried.

By varying the residence time of the pigment in NMP (form 1 minute to 12 hours) the new colouristic-properties are obtained, i.e. the colour in remisson is varying form green to brown and then black depending on the crystal length. In all cases, the colour in transmission remains strongly violet.

The production process of the present invention makes it possible to produce C.I. Pigment Violet 37 in rod-like form reproducibly in a simple manner. C.I. Pigment Violet 37 in rod-like form of the present invention exhibits a specific shape, a color flop effect and/or different colors in remission/transmission, when incorporated in plastics, paints, printing inks etc. and is easy to disperse.

The C.I. Pigment Violet 37 in rod-like form of the present invention is very useful for many purposes, such as coloring plastics, glasses, ceramic products, decorative cosmetic preparations and especially coatings and inks, including printing inks, including security printing inks. All industrially customary printing processes are suitable, for example screen printing, intaglio printing, bronze printing, flexographic printing and offset printing. It is possible to combine two colours (violet/green) or (violet/brown) in the matrix of the application medium using only one pigment, which effect is considered as unique.

For these applications, the pigments of the present invention are also advantageously usable in admixture with transparent and hiding white, color and black pigments and also conventional luster pigments based on metal oxide-coated mica and metal pigments and known (goniochromatic) luster pigments.

The pigments according to the invention can be used for all customary purposes, for example for colouring polymers in the mass, surface coatings (including effect finishes, including those for the automotive sector) and printing inks, and also, for example, for applications in cosmetics. Such applications are known from reference works, for example “Industrielle Organische Pigmente” (W. Herbst and K. Hunger, VCH Verlagsgesellschaft mbH, Weinheim/New York, 2nd, completely revised edition, 1995).

The high molecular weight organic material for the pigmenting of which the pigments or pigment compositions according to the invention may be used may be of natural or synthetic origin. High molecular weight organic materials usually have molecular weights of about from 10³ to 10⁸ g/mol or even more. They may be, for example, natural resins, drying oils, rubber or casein, or natural substances derived therefrom, such as chlorinated rubber, oil-modified alkyd resins, viscose, cellulose ethers or esters, such as ethylcellulose, cellulose acetate, cellulose propionate, cellulose acetobutyrate or nitrocellulose, but especially totally synthetic organic polymers (thermosetting plastics and thermoplastics), as are obtained by polymerisation, polycondensation or polyaddition. From the class of the polymerisation resins there may be mentioned, especially, polyolefins, such as polyethylene, polypropylene or polyisobutylene, and also substituted polyolefins, such as polymerisation products of vinyl chloride, vinyl acetate, styrene, acrylonitrile, acrylic acid esters, methacrylic acid esters or butadiene, and also copolymerisation products of the said monomers, such as especially ABS or EVA.

From the series of the polyaddition resins and polycondensation resins there may be mentioned, for example, condensation products of formaldehyde with phenols, so-called phenoplasts, and condensation products of formaldehyde with urea, thiourea or melamine, so-called aminoplasts, and the polyesters used as surface-coating resins, either saturated, such as alkyd resins, or unsaturated, such as maleate resins; also linear polyesters and polyamides, polyurethanes or silicones.

The said high molecular weight compounds may be present singly or in mixtures, in the form of plastic masses or melts. They may also be present in the form of their monomers or in the polymerised state in dissolved form as film-formers or binders for surface coatings or printing inks, such as, for example, boiled linseed oil, nitrocellulose, alkyd resins, melamine resins and urea-formaldehyde resins or acrylic resins.

Depending on the intended purpose, it has proved advantageous to use the pigments or pigment compositions according to the invention as toners or in the form of preparations. Depending on the conditioning method or intended application, it may be advantageous to add certain amounts of texture-improving agents to the pigment before or after the conditioning process, provided that this has no adverse effect on use of the (effect) pigments for colouring high molecular weight organic materials, especially polyethylene. Suitable agents are, especially, fatty acids containing at least 18 carbon atoms, for example stearic or behenic acid, or amides or metal salts thereof, especially magnesium salts, and also plasticisers, waxes, resin acids, such as abietic acid, rosin soap, alkylphenols or aliphatic alcohols, such as stearyl alcohol, or aliphatic 1,2-dihydroxy compounds containing from 8 to 22 carbon atoms, such as 1,2-dodecanediol, and also modified colophonium maleate resins or fumaric acid colophonium resins. The texture-improving agents are added in amounts of preferably from 0.1 to 30% by weight, especially from 2 to 15% by weight, based on the end product.

The pigments according to the invention can be added in any tinctorially effective amount to the high molecular weight organic material being pigmented. A pigmented substance composition comprising a high molecular weight organic material and from 0.01 to 80% by weight, preferably from 0.1 to 30% by weight, based on the high molecular weight organic material, of an pigment according to the invention is advantageous. Concentrations of from 1 to 20% by weight, especially of about 10% by weight, can often be used in practice.

High concentrations, for example those above 30% by weight, are usually in the form of concentrates (“masterbatches”) which can be used as colorants for producing pigmented materials having a relatively low pigment content, the pigments according to the invention having an extraordinarily low viscosity in customary formulations so that they can still be processed well.

For the purpose of pigmenting organic materials, the pigments according to the invention may be used singly. It is, however, also possible, in order to achieve different hues or colour effects, to add any desired amounts of other colour-imparting constituents, such as white, coloured, black or effect pigments, to the high molecular weight organic substances in addition to the (effect) pigments according to the invention. When coloured pigments are used in admixture with the (effect) pigments according to the invention, the total amount is preferably from 0.1 to 10% by weight, based on the high molecular weight organic material. The pigmenting of high molecular weight organic substances with the pigments according to the invention is carried out, for example, by admixing such a pigment, where appropriate in the form of a masterbatch, with the substrates using roll mills or mixing or grinding apparatuses. The pigmented material is then brought into the desired final form using methods known per se, such as calendering, compression moulding, extrusion, coating, pouring or injection moulding. Any additives customary in the plastics industry, such as plasticisers, fillers or stabilisers, can be added to the polymer, in customary amounts, before or after incorporation of the pigment. In particular, in order to produce non-rigid shaped articles or to reduce their brittleness, it is desirable to add plasticisers, for example esters of phosphoric acid, phthalic acid or sebacic acid, to the high molecular weight compounds prior to shaping.

For pigmenting surface coatings and printing inks, the high molecular weight organic materials and the pigments according to the invention, where appropriate together with customary additives such as, for example, fillers, other pigments, siccatives or plasticisers, are finely dispersed or dissolved in the same organic solvent or solvent mixture, it being possible for the individual components to be dissolved or dispersed separately or for a number of components to be dissolved or dispersed together, and only thereafter for all the components to be brought together.

Dispersing an pigment according to the invention in the high molecular weight organic material being pigmented, and processing a pigment composition according to the invention, are preferably carried out subject to conditions under which only relatively weak shear forces occur so that the effect pigment is not broken up into smaller portions.

The colorations obtained, for example in plastics, surface coatings or printing inks, especially in surface coatings or printing inks, more especially in surface coatings, are distinguished by excellent properties, especially by extremely high saturation, outstanding fastness properties, angle dependent color change (flop) and different colors in remission and transmission, when used in plastics.

When the high molecular weight material being pigmented is a surface coating, it is especially a specialty surface coating, very especially an automotive finish.

Owing to its uncopyable optical effects, the pigment according to the invention is advantageously used for the production of forgery-proof materials from paper and plastic. The term forgery-proof materials made from paper is taken to mean, for example, documents of value, such as banknotes, cheques, tax stamps, postage stamps, rail and air tickets, lottery tickets, gift certificates, entry cards, forms and shares. The term forgery-proof materials made from plastic is taken to mean, for example, cheque cards, credit cards, telephone cards and identity cards. In order to obtain an optimum optical effect, it should be ensured during processing that the rod-like shaped pigment is well oriented, i.e. is aligned as parallel as possible to the surface of the respective medium. This parallel orientation of the pigment particles is best carried out from a flow process, and is generally achieved in all known methods of plastic processing, painting, coating and printing.

The pigments according to the invention are also suitable for making-up the lips or the skin and for colouring the hair or the nails.

The invention accordingly relates also to a cosmetic preparation or formulation comprising from 0.0001 to 90% by weight a pigment according to the invention and from 10 to 99.9999% of a cosmetically suitable carrier material, based on the total weight of the cosmetic preparation or formulation.

Such cosmetic preparations or formulations are, for example, lipsticks, blushers, foundations, nail varnishes and hair shampoos.

The pigments may be used singly or in the form of mixtures. It is, in addition, possible to use pigments according to the invention together with other pigments and/or colorants, for example in combinations as described hereinbefore or as known in cosmetic preparations.

The cosmetic preparations and formulations according to the invention preferably contain the pigment according to the invention in an amount from 0.005 to 50% by weight, based on the total weight of the preparation.

Suitable carrier materials for the cosmetic preparations and formulations according to the invention include the customary materials used in such compositions.

The cosmetic preparations and formulations according to the invention may be in the form of, for example, sticks, ointments, creams, emulsions, suspensions, dispersions, powders or solutions. They are, for example, lipsticks, mascara preparations, blushers, eye-shadows, foundations, eyeliners, powder or nail varnishes.

If the preparations are in the form of sticks, for example lipsticks, eye-shadows, blushers or foundations, the preparations consist for a considerable part of fatty components, which may consist of one or more waxes, for example ozokerite, lanolin, lanolin alcohol, hydrogenated lanolin, acetylated lanolin, lanolin wax, beeswax, candelilla wax, microcrystalline wax, carnauba wax, cetyl alcohol, stearyl alcohol, cocoa butter, lanolin fatty acids, petrolatum, petroleum jelly, mono-, di- or tri-glycerides or fatty esters thereof that are solid at 25° C., silicone waxes, such as methyloctadecane-oxypolysiloxane and poly(dimethylsiloxy)-stearoxysiloxane, stearic acid monoethanolamine, colophane and derivatives thereof, such as glycol abietates and glycerol abietates, hydrogenated oils that are solid at 25° C., sugar glycerides and oleates, myristates, lanolates, stearates and dihydroxystearates of calcium, magnesium, zirconium and aluminium.

The fatty component may also consist of a mixture of at least one wax and at least one oil, in which case the following oils, for example, are suitable: paraffin oil, purcelline oil, perhydrosqualene, sweet almond oil, avocado oil, calophyllum oil, castor oil, sesame oil, jojoba oil, mineral oils having a boiling point of about from 310 to 410° C., silicone oils, such as dimethylpolysiloxane, linoleyl alcohol, linolenyl alcohol, oleyl alcohol, cereal grain oils, such as wheatgerm oil, isopropyl lanolate, isopropyl palmitate, isopropyl myristate, butyl myristate, cetyl myristate, hexadecyl stearate, butyl stearate, decyl oleate, acetyl glycerides, octanoates and decanoates of alcohols and polyalcohols, for example of glycol and glycerol, ricinoleates of alcohols and polyalcohols, for example of cetyl alcohol, isostearyl alcohol, isocetyl lanolate, isopropyl adipate, hexyl laurate and octyl dodecanol.

The fatty components in such preparations in the form of sticks may generally constitute up to 99.91% by weight of the total weight of the preparation.

The cosmetic preparations and formulations according to the invention may additionally comprise further constituents, such as, for example, glycols, polyethylene glycols, polypropylene glycols, monoalkanolamides, non-coloured polymeric, inorganic or organic fillers, preservatives, UV filters or other adjuvants and additives customary in cosmetics, for example a natural or synthetic or partially synthetic di- or tri-glyceride, a mineral oil, a silicone oil, a wax, a fatty alcohol, a Guerbet alcohol or ester thereof, a lipophilic functional cosmetic active ingredient, including sun-protection filters, or a mixture of such substances.

A lipophilic functional cosmetic active ingredient suitable for skin cosmetics, an active ingredient composition or an active ingredient extract is an ingredient or a mixture of ingredients that is approved for dermal or topical application. The following may be mentioned by way of example:
active ingredients having a cleansing action on the skin surface and the hair; these include all substances that serve to cleanse the skin, such as oils, soaps, synthetic detergents and solid substances;
active ingredients having a deodorising and perspiration-inhibiting action: they include antiperspirants based on aluminium salts or zinc salts, deodorants comprising bactericidal or bacteriostatic deodorising substances, for example triclosan, hexachlorophene, alcohols and cationic substances, such as, for example, quaternary ammonium salts, and odour absorbers, for example ®Grillocin (combination of zinc ricinoleate and various additives) or triethyl citrate (optionally in combination with an antioxidant, such as, for example, butyl hydroxytoluene) or ion-exchange resins;
active ingredients that offer protection against sunlight (UV filters): suitable active ingredients are filter substances (sunscreens) that are able to absorb UV radiation from sunlight and convert it into heat; depending on the desired action, the following light-protection agents are preferred: light-protection agents that selectively absorb sunburn-causing high-energy UV radiation in the range of approximately from 280 to 315 nm (UV-B absorbers) and transmit the longer-wavelength range of, for example, from 315 to 400 nm (UV-A range), as well as light-protection agents that absorb only the longer-wavelength radiation of the UV-A range of from 315 to 400 nm (UV-A absorbers); suitable light-protection agents are, for example, organic UV absorbers from the class of the p-aminobenzoic acid derivatives, salicylic acid derivatives, benzophenone derivatives, dibenzoylmethane derivatives, diphenyl acrylate derivatives, benzofuran derivatives, polymeric UV absorbers comprising one or more organosilicon radicals, cinnamic acid derivatives, camphor derivatives, trianilino-s-triazine derivatives, phenyl-benzimidazolesulfonic acid and salts thereof, menthyl anthranilates, benzotriazole derivatives, and/or an inorganic micropigment selected from aluminium oxide- or silicon dioxide-coated TiO₂, zinc oxide or mica;
active ingredients against insects (repellents) are agents that are intended to prevent insects from touching the skin and becoming active there; they drive insects away and evaporate slowly; the most frequently used repellent is diethyl toluamide (DEET); other common repellents will be found, for example, in “Pflegekosmetik” (W. Raab and U. Kindl, Gustav-Fischer-Verlag Stuttgart/New York, 1991) on page 161;
active ingredients for protection against chemical and mechanical influences: these include all substances that form a barrier between the skin and external harmful substances, such as, for example, paraffin oils, silicone oils, vegetable oils, PCL products and lanolin for protection against aqueous solutions, film-forming agents, such as sodium alginate, triethanolamine alginate, polyacrylates, polyvinyl alcohol or cellulose ethers for protection against the effect of organic solvents, or substances based on mineral oils, vegetable oils or silicone oils as “lubricants” for protection against severe mechanical stresses on the skin;
moisturising substances: the following substances, for example, are used as moisture-controlling agents (moisturisers): sodium lactate, urea, alcohols, sorbitol, glycerol, propylene glycol, collagen, elastin and hyaluronic acid;
active ingredients having a keratoplastic effect: benzoyl peroxide, retinoic acid, colloidal sulfur and resorcinol;
antimicrobial agents, such as, for example, triclosan or quaternary ammonium compounds;
oily or oil-soluble vitamins or vitamin derivatives that can be applied dermally: for example vitamin A (retinol in the form of the free acid or derivatives thereof), panthenol, pantothenic acid, folic acid, and combinations thereof, vitamin E (tocopherol), vitamin F; essential fatty acids; or niacinamide (nicotinic acid amide);
vitamin-based placenta extracts: active ingredient compositions comprising especially vitamins A, C, E, B₁, B₂, B₆, B₁₂, folic acid and biotin, amino acids and enzymes as well as compounds of the trace elements magnesium, silicon, phosphorus, calcium, manganese, iron or copper;
skin repair complexes: obtainable from inactivated and disintegrated cultures of bacteria of the bifidus group;
plants and plant extracts: for example arnica, aloe, beard lichen, ivy, stinging nettle, ginseng, henna, camomile, marigold, rosemary, sage, horsetail or thyme;
animal extracts: for example royal jelly, propolis, proteins or thymus extracts;
cosmetic oils that can be applied dermally: neutral oils of the Miglyol 812 type, apricot kernel oil, avocado oil, babassu oil, cottonseed oil, borage oil, thistle oil, groundnut oil, gamma-oryzanol, rosehip-seed oil, hemp oil, hazelnut oil, blackcurrant-seed oil, jojoba oil, cherry-stone oil, salmon oil, linseed oil, cornseed oil, macadamia nut oil, almond oil, evening primrose oil, mink oil, olive oil, pecan nut oil, peach kernel oil, pistachio nut oil, rape oil, rice-seed oil, castor oil, safflower oil, sesame oil, soybean oil, sunflower oil, tea tree oil, grapeseed oil or wheatgerm oil.

The preparations in stick form are preferably anhydrous but may in certain cases comprise a certain amount of water which, however, in general does not exceed 40% by weight, based on the total weight of the cosmetic preparation.

If the cosmetic preparations and formulations according to the invention are in the form of semi-solid products, that is to say in the form of ointments or creams, they may likewise be anhydrous or aqueous. Such preparations and formulations are, for example, mascaras, eyeliners, foundations, blushers, eye-shadows, or compositions for treating rings under the eyes.

If, on the other hand, such ointments or creams are aqueous, they are especially emulsions of the water-in-oil type or of the oil-in-water type that comprise, apart from the pigment, from 1 to 98.8% by weight of the fatty phase, from 1 to 98.8% by weight of the aqueous phase and from 0.2 to 30% by weight of an emulsifier.

Such ointments and creams may also comprise further conventional additives, such as, for example, perfumes, antioxidants, preservatives, gel-forming agents, UV filters, colorants, pigments, pearlescent agents, non-coloured polymers as well as inorganic or organic fillers. If the preparations are in the form of a powder, they consist substantially of a mineral or inorganic or organic filler such as, for example, talcum, kaolin, starch, polyethylene powder or polyamide powder, as well as adjuvants such as binders, colorants etc.

Such preparations may likewise comprise various adjuvants conventionally employed in cosmetics, such as fragrances, antioxidants, preservatives etc.

If the cosmetic preparations and formulations according to the invention are nail varnishes, they consist essentially of nitrocellulose and a natural or synthetic polymer in the form of a solution in a solvent system, it being possible for the solution to comprise other adjuvants, for example pearlescent agents.

In that embodiment, the coloured polymer is present in an amount of approximately from 0.1 to 5% by weight.

The cosmetic preparations and formulations according to the invention may also be used for colouring the hair, in which case they are used in the form of shampoos, creams or gels that are composed of the base substances conventionally employed in the cosmetics industry and a pigment according to the invention.

The cosmetic preparations and formulations according to the invention are prepared in conventional manner, for example by mixing or stirring the components together, optionally with heating so that the mixtures melt.

Various features and aspects of the present invention are illustrated further in the examples that follow. While these examples are presented to show one skilled in the art how to operate within the scope of this invention, they are not to serve as a limitation on the scope of the invention where such scope is only defined in the claims. Unless otherwise indicated in the following examples and elsewhere in the specification and claims, all parts and percentages are by weight, temperatures are in degrees centigrade and pressures are at or near atmospheric.

### EXAMPLES

The analytical samples for measuring the hue-value h and the saturation C* and also the hiding power ΔE*-S/W are prepared according to DIN 53 775 part 7 using pressed PVC plates (1 mm thick) containing 1.0% by weight of the C.I. Pigment Violet 37 in rod-like form.

All colour measurements in remission/transmission are effected using a Minolta CM 3610d spectrophotometer (d/8 geometry, including the gloss, illuminant D65, observer 10°) and B&W Leneta cards. All “angle-depending” measurements are effected using a Datacolor FX 10 and B&W Leneta cards.

### Examples

### Example 1

10 g of crude C.I. Pigment Violet 37 are dispersed in 100 ml of N-methyl-pyrrolidone (NMP) at room temperature. The mixture is then heated up to 190° C. using an oil-bath and this temperature is kept for 15 minutes. The reaction is cooled down to 30° C. and the newly formed crystals are isolated by filtration. The NMP remaining in the pigment is removed by washing the filter-cake using 100 ml of water. The pigment is then dried in a vacuum oven at 60° C.

By dispersing 1% of the dried powder in 99% PVC by warm-calendaring (160° C.; cf. application example 1), a green colour is observed in remission; in transmission the effect is violet.

By varying the residence time of the pigment at high temperature other colours are observed.

| Residence Time | Colour in Remission | Colour in Transmission |
|---|---|---|
| 1 minute | light-green | violet |
| 15 Minutes | dark-green | violet |
| 3 hours | brown | violet |
| 12 hours | black | violet |

### Application Example 1

### Measurement of the Color of 0.35 Mm Rolled PVC Sheets:

Premix:

0.4 g Pigment is mixed for 30 minutes with 14.0 g of a base mixture and then slowly stirred with 26.0 g of polyvinyl chloride (PVC) (EVIPOL® SH 7020, EVC GmbH). The base mixture consists of plastiziser (12.9 g Palatinol® 10P (di-2-propylheptylphthalate, BASF), 0.6 g Drapex® 39 (epoxidised soya bean oil, Witco Vinyl Additives GmbH) and 0.5 g Mark BZ 561 (Crompton Vinyl Additives GmbH).

Production of Rolled Sheets:

The mixture of PVC and pigment/base mixture obtained above is rolled in a 2-roll mill (Collin model, D-85560 Ebersberg) at a roll temperature of 160° C. (each roll) in accordance with the following:

a) hot-rolling for 6 minutes (rolled sheet turned every minute, roll nip 0.35 mm).

1. A C.I. Pigment Violet 37 in rod-like form.

2. The C.I. Pigment Violet 37 in rod-like form according to claim 1 having a length of 0.5 to 30 μm, a width of 0.05 to 1 μm, and an average thickness of 0.01 to 0.200 μm.

3. A process for the preparation of a C.I. Pigment Violet 37 in rod-like form, which comprises
(a) dispersing and partially dissolving crude C.I. Pigment Violet 37 in a polar solvent at elevated temperatures for a defined period of time, wherein said polar solvent is selected from the group consisting of N,N,N′,N′-tetramethylurea, N,N-dimethylacetamide, N-methylformamide, N,N-dimethylformamide (DMF), and N-methyl-pyrrolidone (NMP) and wherein elevated temperatures mean temperatures from 100° C. to reflux temperature of the solvent to produce a C.I. Pigment Violet 37 in rod-like form having a length of 0.5 to 30 μm, a width of 0.05 to 1 μm and an average thickness of 0.01 to 0.200 μm.

4. The process according to claim 3, wherein the solvent is N-methyl-pyrrolidone.

5. The process according to claim 4, wherein the pigment is heated up to 120° C. to reflux temperature in step (a).

6. The process according to claim 3, wherein; the molar ratio of the crude C.I. Pigment Violet 37 to solvent is 1:133 to 1:33.

7. A C.I. Pigment Violet 37 in rod-like form obtained according to the process claim 3.

8. A high molecular weight organic material containing an effective coloring amount of the C.I. Pigment Violet 37 in rod-like form according to claims 1.

9. A method for coloring high molecular weight organic material which comprises incorporating an effective coloring amount of the C.I. Pigment Violet 37 in rod-like form according to claim 1 into said organic material.

10. A forgery-proof material, comprising the C.I. Pigment Violet 37 in rod-like form according to claim 1.

11. The C.I. Pigment Violet 37 in rod-like form according to claim 2 having a length of 0.5 to 30 μm, a width of 0.100 to 0.500 μm and an average thickness of 0.03 to 0.100 μm.

12. A high molecular weight organic material containing an effective coloring amount of the C.I. Pigment Violet 37 in rod-like form according to claim 2.

13. A high molecular weight organic material containing an effective coloring amount of the C.I. Pigment Violet 37 in rod-like form according to claim 7.

14. A method for coloring high molecular weight organic material which comprises incorporating an effective coloring amount of the C.I. Pigment Violet 37 in rod-like form according to claim 2 into said organic material.

15. A method for coloring high molecular weight organic material which comprises incorporating an effective coloring amount of the C.I. Pigment Violet 37 in rod-like form according to claim 7 into said organic material.

16. A forgery-proof material, comprising the C.I. Pigment Violet 37 in rod-like form according to claim 2.

17. A forgery-proof material, comprising the C.I. Pigment Violet 37 in rod-like form according to claim 7.
